(19) United States
(12) Patent Application Publication
(10) Pub. No.: US 2004/0116903 A1
(43) Pub. Date: Jun. 17, 2004

(54) Spray pump wound irrigation device

(76) Inventor: Ali Mohammed Osman, Peoria, IL (US)

Correspondence Address: JoAnne M. Denison, Suite 1608, 212 W. Washington Blvd., Chicago, IL, 60606-3487, US

(21) Appl. No.: 10/316,332

(22) Filed: Dec. 11, 2002

(51) Int. Cl. A61M 27/00

(52) U.S. Cl. 604/543

(57) ABSTRACT

The present invention consists of a hand pump type of device for irrigation or cleansing of wounds or laceration comprising a hand held pumping device, a back splash shield secured to a fluid nozzle, elongate clear plastic tubing connecting the hand held pumping device to a bottle of medicinal fluid for cleansing the wound or laceration. With the present invention, the user of this device merely opens up a sterile package, removes the device, removes the top from a standard sized bottle of medicinal fluid, typically isotropic saline solution, and screws on a cap or top having a dip tube extending adjacent the bottom of the bottle of fluid, and then begins to simply and easily pump away until the desired amount of fluid has been delivered to the wound site to cleanse it. Both the nozzle and the pumping mechanism have been designed so that the pressure and spray pattern of fluid being delivered to the wound are in the optimal range of substantially four to fifteen psi.

### FIELD OF THE INVENTION

The present invention relates to the field of spray type irrigation devices, and more particularly those which use a hand pump sprayer to cleanse an open wound or laceration.

### BACKGROUND OF THE INVENTION

Currently, in the marketplace, there are available a wide variety of devices which are suitable for use in cleansing or irrigating a wound by spraying a solution over the wound. Cleansing of a wound is generally a cumbersome and tedious process which requires that medical personnel fill and refill large syringes multiple times with fluid to achieve effective wound cleansing. The fluid generally used is an isotonic saline and it must be delivered in fairly substantial quantities at a desired pressure. Current clinical guidelines indicate than an irrigation pressure of four to fifteen pounds per square inch is effective for the cleansing of wounds or lacerations before repair. The amount of pressure is sufficient for removal of foreign particles, debris and bacteria to thereby promote healing and minimization of infection or inflammation, yet low enough to avoid or minimize damage to regenerating granulation tissue in the wound. The guidelines also recommend the use of high pressure irrigation for mechanical debridement to remove devitalized or necrotic tissues.

Delivery of irrigation fluid to the wound site is usually accomplished by pumping liquid from a hypodermic syringe through an attached needle, or catheter which has been directed toward the wound site. Most of the liquid effluent which has passed over the wound site is collected in some manner, either into a bowl or with absorbent material. However, some back splash of fluid and debris from the would occurs, especially at higher pressures of delivery. The back splash contains not only the irrigation liquid, but it also contains fluid from the wound and loose matter extracted from the wound, particularly in a situation where wound irrigation is conducted for debridement purposes. Hence, the use of a shield of some sort is generally necessary and desirable. The current clinical practice guidelines of four to fifteen pounds per square inch of fluid pressure on the wound site for effective cleansing of wounds is sometimes difficult to achieve for new or inexperienced practitioners. It leaves much up to the judgement of the particular person cleansing the wound. The failure to irrigate the wound at the appropriate fluid pressure can adversely affect the health and healing of the wound and may result in infection of the wound or laceration. Thus, fluid delivered at too high a pressure may effectively lodge extraneous particulates deeper into tissues, rather than wash them away from the area, while fluid delivered at too low of a pressure may produce an ineffective cleansing of the wound.

In a typical situation, the care giver must depress with the thumb and fingers the plunger of a 35 cc hypodermic syringe filled with irrigation fluid and fitted with a 19 gauge needle of approximately 1.5″ in length to produce an 8 psi delivery of irrigation fluid. But this assumes that all care givers have the manual dexterity and strength to effectively depress the plunger of the hypodermic syringe. Moreover, since the plungers are difficult to depress over and over again without some fatigue being involved, it is likely that at the end of the process the pressure might be insufficient to effectuate a thorough cleansing of the wound site.

It should also be noted that while there have been found in the prior art a wide variety of delivery devices, such as spray bottles, squeeze bottles, aerosol canisters or water wands, none of these seems well adapted to be fitted effectively with a back splash shield, which would protect the patient and care giver alike from extraneous fluid flowing about or the fine particulate aerosol mist created during wound irrigation. Thus, it is most important to develop an easy to use hand pump type of device which requires less manual dexterity and strength than the filling and refilling of a large syringe. It is also very important to develop a wound irrigation device that can be easily and simply outfitted with a wide variety of back splash shields, depending on the size and type of wound involved.

Various devices are seen in design patents. For example, U.S. Des. Pat. No. 333,000 discloses a spray bottle with an elongated nozzle and a conical fluid retainment chamber disposed beneath. U.S. Des. Pat. Nos. 345,016 and 344,133 discloses a high domed transparent splash shield which prevents back splash from wound irrigation from spraying on areas other than the wound and its immediate surrounding area. U.S. Pat. No. 5,496,290 discloses the use of a rather large pie pan shaped splash shield for wound irrigation. A similar design for a splash shield which is bell shaped in design is disclosed in U.S. pat. No. 4,769,003. Splash shields may also protect the patient from injury from a hypodermic needle used during irrigation, when it is used in too close proximity to the wound.

U.S. Pat. Nos. 4,413,987 and 4,421,505 discloses the use of an irrigation system wherein a narrow piece of tubing is inserted into an body part and fluid is supplied to the wound at a controlled rate, while at the opposite end of the wound a vacuum supplied drainage tube is provided to suck spent fluid out of the wound. U.S. Pat. No. 4,778,446 discloses the use of a wound irrigation system which has a square wound cover attached to a collar which is adapted to be removably sealed by means of a wide cap. The wide cap may be provided with an opening to receive a tube used to provide a flow of fluid to the wound therebelow. A drainage tube to the device for the wound effluent is further provided.

In U.S. Pat. No. 5,071,104 is disclosed a wound irrigation system having two bladders for containing fluid. One bladder is a pressure bladder to control the flow of fluid found in the second bladder. The fluid bladder is attached to tubing and the flow of fluid over the wound is controlled by a manually operated valve. Similarly, in U.S. Pat. No. 5,133,701 is disclosed a disposable pressure wound irrigation devise with a first chamber containing cleansing solution and a second chamber containing the fluid propellant and a pressure relief valve to control the flow of fluid from the device. U.S. Pat. No. 5,441,174 discloses the use of portable wound irrigation device having an inflatable bag that expands as fluid fills the device and the elasticized walls of the bag produce pressure to provide a properly pressurized fluid flow over the wound to be cleansed.

Another device utilized during gastric lavage or wound irrigation is shown in U.S. Pat. No. 5,795,324 which discloses a threaded cap member which is adopted to threadedly secure to a saline bottle and is further connected to a syringe tip which may be inserted into a length of tubing which provides an evacuation route for the fluid over the wound or into the stomach portion of a patient. In U.S. Pat. Nos. 5,830,197 and 6,468,253 is disclosed a device which provides a fluid receptacle with a nozzle having a plurality of slits or circular apertures disposed therein to supply a stream of cleansing fluid at the desire pressure and area of dispersion by simply squeezing the fluid receptacle. However this device can be hard to hold and difficult to use when the bottle of fluid is large and full of fluid. Moreover, the user must wait for ingress of air after a quantity of fluid has been dispelled before squeezing out more cleansing fluid over the wound to be treated.

U.S. Pat. No. 5,860,947 discloses the use of a hypodermic syringe with a two way syringe fitted with a check valve which may be filled via a short fill stem which has been dipped into a sterile basin of fluid to be used during irrigation of a wound. With this particular device, the user must still open a bottle of saline and fill a sterile basin with fluid to use the device. U.S. Pat. No. 5,931,820 discloses a device for irrigating wounds and for gastric lavage which utilized a connector with a spiked end which is adopted to be received into a self-sealing outlet of a compressible bag of cleansing fluid which fluid may the be dispersed to the wound via an elongate tube.

U.S. Pat. No. 5,941,859 discloses the use of a specialized domed shaped splash shield which is provided with a high pressure nozzle and an aperture for effluent disposed after washing a wound site. U.S. Pat. Nos. 6,050,981 and 6,093,182 disclose the use of a hypodermic device which is threadedly secured to a square wound cover which may be used to irrigate a wound. The device may also be provided with a pump type device, yet the pump is only connected to a bladder containing fluid.

U.S. Pat. No. 6,186,988 discloses a wound irrigation system having a housing with a water filter and an antibiotic, which is connected between a water supply and a hand spray unit. A water temperature display is also provided to assist in mixing the proper temperature of water directed to flow into the wound area.

U.S. Pat. No. 6,293,929 discloses a specialized splash shield having two components, with a more rigid first portion connected to the fluid source and being domed shape in configuration, and a second portion being more flexible and being ring shaped and fitting onto the end of the first rigid portion of the splash shield. Similarly in U.S. Pat. No. 6,402,724 is disclosed a fluid irrigation system with a two portion splash shield wherein the domed portion of the shield is more rigid while the ring shaped second portion is flexible and body conforming to retain fluid within the splash shield. A drain tube is provided along the rim portion of the device. U.S. Pat. No. 6,332,876 discloses a compressible syringe which is provided with bellows with the rearward frusto-conical bellow walls being thicker than the forward frusto-conical bellow walls. Such a bellows type syringe may be utilized for irrigation of wounds.

U.S. Pat. No. 6,394,996 discloses the use of an irrigation system for debriding a tract wound which is provided with an elongate tip with a flexible shaft which may be inserted into the deepest part of a tract wound. Flow of fluid to the wound is controlled by a hand held valve with a hand grip and trigger mechanism. In U.S. Pat. No. 6,458,109 is disclosed the use of a bandage type apparatus which includes a nebulizer with treatment fluid flowing to the bandage, a temperature control means to control the temperature of the fluid being applied to the wound, and a drainage tube for effluent.

Thus, nowhere in the prior is seen a simple, easy to use trigger type wound irrigation spray device that delivers a controlled spray of pressurized fluid over a wound, which is provided with an elongate tube connected to a threaded cap and dip tube, wherein the cap may be threaded onto a standard medical fluid solution bottles which are commonly available in a variety of sizes, including 1 liter, 500 ml or 250 ml. It is intented that the present invention will fit all sizes of standard medical solution bottles. The present invention is especially directed towards the cleansing of fresh wounds and lacerations.

### SUMMARY OF THE INVENTION

The present invention consists of a simple to use, cost effective and completely disposable wound irrigation system. In the present invention, a hand pump spray assembly is connected to a portion of clear plastic tubing which is in turn connected to a threaded cap which may be removably secured to any standard size bottle containing a lavage or cleansing fluid, such as a saline solution. The hand pump may be provided with a nozzle portion which directs a spray flow to the wound at the desired pressure. If desired, a rotating nozzle may be provided with a variety of spray patterns and nozzle openings for various sizes and types of wounds to be cleansed.

In one preferred embodiment of the present invention, the entire spray pump and tubing is made from plastic or other copolymer and is placed in a completely sealed, yet easy to open plastic pouch or container. The user simply opens up the package, then opens up a bottle of cleansing fluid and screws on the cap of the inventive device onto the bottle opening. In such a manner, a large quantity of fluid, even the entire bottle of fluid, may be easily and rapidly delivered at a predetermined pressure and spray pattern onto the wound area.

Of course, the pump nozzle may be outfitted with a snap on type of splash shield to prevent back splash onto the patient or the surrounding area, or the back splash shield may be readily removable when supplied with the device, or it may even be supplied with the device as an integral, non-removable part of the device. In such a manner a wide variety of sizes and shapes of spray shields may be used with the present invention depending upon the size and type of wound to be treated.

### OBJECTS OF THE INVENTION

Thus, it is one primary object of the present invention to provide a simple hand held device having a fluid pumping trigger which may quickly and easily deliver a large quantity of fluid by simply squeezing the trigger and dispelling the fluid over a wound or lacertation at a predetermined pressure.

It is yet an additional primary object of the present invention to provide a simple hand held device having a fluid pumping trigger communicating with an elongate portion of plastic tubing and a threaded cap, which threaded cap is adapted to secure onto the top collar of a standard bottle of medical cleansing fluid, including 1 liter, 500 ml and 250 ml sized bottles.

It is a further primary object of the present invention to provide a simple hand held fluid pumping device which communicates with a tube extending through a cap and into a cleansing fluid bottle directly adjacent the bottom of the bottle to effectively draw out all the cleansing fluid by simply pumping the trigger of the device.

It is still an additional primary object of the present invention to provide a simple hand held fluid pumping device which may be sold in separate sterile packages and is intended to be disposable so that the user need only tear open the package, remove the cap of a standard bottle of medical cleansing fluid, screw on the cap provided with the present invention to the fluid bottle and then pump away to quickly and efficiently cleanse a wound area at the proper pressure desired.

Yet a further primary object of the present invention is to provide a hand held fluid pumping device which may be provided with a plurality of different types of nozzles on a rotating nozzle assembly wherein the user can easily and simply select from a variety of spray patterns and pressures, depending upon the size and type of the wound to be treated.

These and other objects and advantages of the present invention can be readily derived from the following detailed description of the drawings taken in conjunction with the accompanying drawings present herein and should be considered as within the overall scope of the invention.

### BRIEF DESCRIPTION OF THE DRAWINGS

FIG. 1 is a side perspective view of the present invention showing the inventive device with a spray shield attached thereto and tubing and cap sized for a standard sterile medical solution bottle.

FIG. 2 is a elevation partial cross section of the present invention showing only the spray shield attached and a portion of the lower tubing which would lead into a standard sterile medical solution bottle.

FIG. 3 is a partial cross section of the present invention showing a rotatable nozzle with a plurality of discharge apertures.

FIG. 4 is a front elevation view of a nozzle shown in FIG. 3 having a plurality of discharge apertures.

FIG. 5 is a side perspective of sterile packaging for the present invention, showing the packaging partially opened, with the top peeled back.

### DETAILED DESCRIPTION OF THE DRAWINGS

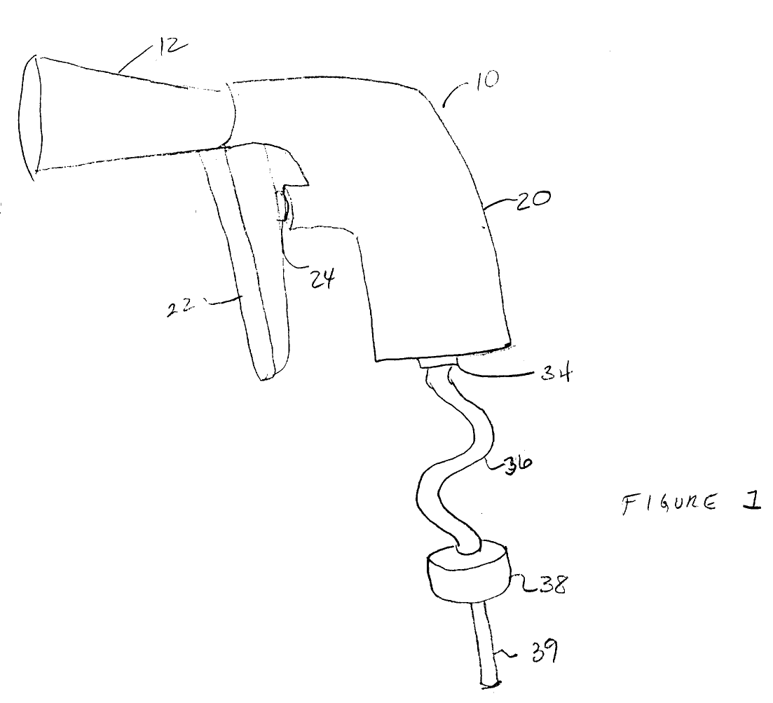

Shown now in FIG. 1 is a cross section of the present invention, wound irrigation hand pump 10 which has been provided with back splash shield 12 which is frusto-conical in shape. It should be noted that a variety of other shapes of back splash shields may be used with the present invention, in that the back splash shield may be clamped onto the device, or it may be slipfitted over the spray nozzle, and all of such modifications should be considered within the overall scope of the present invention. Also shown are hand trigger 22, which communicates with pressurizing piston 24 to pump fluid up into and out of the device through a nozzle contained within back splash shield 12. A bias spring 26 is provided to maintain the trigger 22 in an outward position when the device is not in use, and also to effecutate a vacuum in pressurizing piston 24 to draw fluid up through inlet tubing or tract 28 after trigger 22 has been depressed and released. A housing 20 contains the tubes and spray mechanism for wound irrigation hand pump 10. A connector element 34 connects the housing 20 to the inlet tubing 36 which communicates with threaded cap or top 38. The threaded cap or top 38 is adapted to threadedly secure to the opening of a standard bottle of medical fluid, such as isotropic saline solution, a fluid which is typically utilized to irrigate wounds. Threaded cap or top 38 is made in a standard size so that it is adapted to fit and secure upon the threaded apertures of standard medical fluid bottles which are commonly available in the following sizes: 1 liter, 500 ml and 250 ml sizes. Or, two or more different sizes of threaded caps or tops 38 may be provided at the time of sale of the present invention, so that the user need only select a cap or top 38 and slide it onto the end of inlet tubing 36. A dip tube 39 extends into the bottom portion of the fluid bottle in order to draw as much fluid from the bottle as is possible, and in one preferred embodiment of the present invention, the dip tube 39 may be slidingly retained within the threaded cap or top 38 such that the user of the device may slide the dip tube up or down through the aperture in cap or top 38 to adjust the length of the dip tube 39 so that it lies close as possible to the bottom of the bottle of fluid to be used in the wound cleansing procedure.

In one preferred embodiment of the present invention, the entire assembly of wound irrigation hand pump 10 is composed from plastic or other polymers. Inlet tubing 36 is preferrably composed of clear plastic tubing so that the fluid flow into wound irrigation hand pump 10 is readily viewed by the user of the device. The back splash shield 12 may further be made from a clear plastic to view progress of cleansing of the wound, and it may also be provided with an aperture and a length of clear plastic tubing where it is desired that the effluent be neatly drained into a basin or bottle or other appropriate chamber typically used for the collection of fluids.

FIG. 2 shows a cross section of wound irrigation hand pump 10 wherein in the interior mechanism of the device is readily visible. Connected to back splash shield 12 is a nozzle opening 14 which is in turn connected to nozzle tract or tubing 16. The nozzle opening 14 may be produced in a variety of shapes and configurations suitable to different types of wounds. It is anticipated that the nozzle opening 14 should be configured to produce the optimal wound cleansing pressure of between four and fifteen psi. Larger wounds and larger back splash shields 12 would necessarily demand that the nozzle opening 14 be configured to produce a wider area of coverage, whereas smaller wounds and a smaller back splash shield would demand a more narrow area of coverage produced by nozzle opening 14.

Nozzle tract or tubing 16 communicates directly with pressure chamber 18 wherein fluid pressure accumulates to force fluid out of nozzle opening 14 by means of pressurizing piston 24 which communicates with trigger 22. When trigger 22 is squeezed by the user, check ball 32 seats firmly in check valve 30 to prevent fluid flow to back down plastic inlet tubing 36. Accordingly, fluid is forced up and out nozzle tract or tubing 16 and out through nozzle opening 14 with each squeeze of trigger 22. In such a manner, wound cleansing fluid is quickly and easily dispelled across a wound site at the proper pressure and area of spray. The user of the device does not hove to reload syringe after syringe.

The user may even have some limited dexterity or experience in using such a device to effectuate a proper wound cleansing.

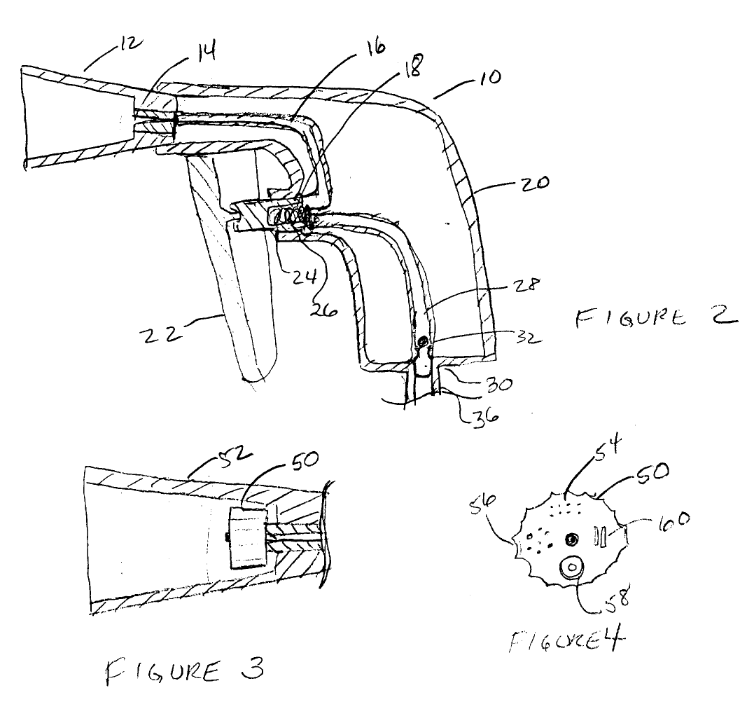

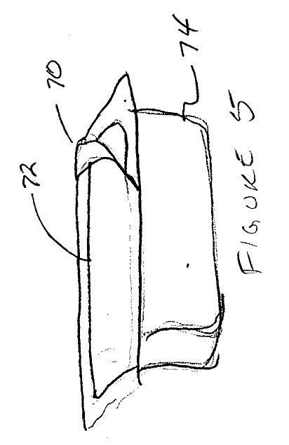

FIG. 3 shows an alternative form of rotating nozzle opening 50 disposed within back splash shield 52 which may be manipulated by the user to one of a plurality of different spray pressures and/or patterns, as desired by the user. FIG. 4 shows four such differing patterns with a variety of hole sizes, from a circular array of pin holes 56 to wide circular openings 58 to sets of slots 60 or even a rectangular spray pattern 54 disposed therein. In such a manner, a simple, effective and easy to use wound irrigation hand pump is shown. Unlike spray bottles and water wands, a slender nozzle makes it easy to attach a wide variety of back splash shields.

FIG. 5 shows a typical sterile packaging assembly for wound irrigation hand pump 10 wherein the core giver could simply peel back peel away cover 72 and extract the wound irrigation hand pump 10 from plastic pan with rim 73. The device is then merely screwed onto the cap of a standard medical solution bottle and the care giver then simply begins to squeeze trigger 22 and pump away, easily and gently cleansing the wound, until the desired amount of solution has been delivered to the wound site. In such a manner, the medical cleansing and/or debridement of wounds becomes a simple, one step process, with minimal hand fatigue.

Although in the foregoing detailed description the present invention has been described by reference to various specific embodiments, it is to be understood that modifications and alterations in the structure and arrangement of those embodiments other than those specifically set forth herein may be achieved by those skilled in the art and that such modifications and alterations are to be considered as within the overall scope of this invention.

What is claimed is:

1. A sterile fluid pumping device having a housing with a fluid pressurization chamber, means for egress of a pressurized fluid, a nozzle for producing a desired spray of pressurized fluid, an inlet tube communicating with the fluid pressurization chamber and attachment means for the pumping device for removably securing the fluid pumping device to a container of sterile fluid.

2. The sterile fluid pumping device of claim 1 further having a trigger which, when depressed, creates fluid pressure within the fluid pressurization chamber and forces pressurized fluid out through the nozzle, producing a spray of pressurized fluid.

3. The sterile fluid pumping device of claim 2 further having an annular back splash shield which is adapted to fit the nozzle in order to prevent pressurized fluid from spraying onto the user of the device or the patient which the device is used upon.

4. The sterile fluid pumping device of claim 2 further having a plurality of nozzle opening patterns which the user may select one from by manipulating the nozzle opening to the desired spray pattern.

5. The sterile fluid pumping device of claim 4 in which the nozzle may be rotated by the user in order to select the desired spray pattern.

6. The sterile fluid pumping device of claim 2 in which the attachment means for the pumping device for removably securing the fluid pumping device to a container of sterile fluid comprises a threaded cap with an aperture in the cap for receiving a length of tubing.

7. The sterile fluid pumping device of claim 6 in which the threaded cap further has a dip tube extending from the threaded cap wherein the dip tube may be slidingly adjusted by the user to extend it to the bottom of a container of fluid.

8. The sterile fluid pumping device of claim 2 which produces a spray of pressurized sterile fluid from the nozzle substantially in the range of pressurization from four to fifteen psi.

9. The sterile fluid pumping device of claim 1 further being provided with sterile packaging to keep the device clean and sterile until the time of use.

10. The sterile fluid pumping device and packaging of claim 9 further having a plastic pan to receive the sterile fluid pumping device, and a peel away cover to secure the device within the sterile packaging until the desired time to use the device.
